(19) United States
(12) Patent Application Publication
(10) Pub. No.: US 2026/0171260 A1
(43) Pub. Date: Jun. 18, 2026

(54) Systems and Methods for Analyzing and Condensing Audio Information Relating to an Encounter

(71) Applicant: OPTUM, INC., Minnetonka, MN (US)

(72) Inventors: Wajhi Ahmed, Schaumburg, IL (US); Arjun Porwal, Uttar Pradesh (IN); Gregory J. Boss, Saginaw, MI (US); Ramprasad Anandam Gaddam, Haryana (IN); Rafael Campos Do Amaral e Vasconcellos, Plymouth, MN (US)

(21) Appl. No.: 18/978,755

(22) Filed: Dec. 12, 2024

(51) Int. Cl. G16H 50/70; G06F 40/289; G10L 15/06; G10L 15/183; G10L 25/66

(52) U.S. Cl. CPC G16H 50/70; G06F 40/289; G10L 15/063; G10L 15/183; G10L 25/66

(57) ABSTRACT

The present disclosure generally relates to generating a summary for an encounter between an individual and a provider of medical services. More specifically, the disclosure relates to systems and methods for generating a concise and dynamic summary related to a reason for the encounter by accurately and efficiently curating and analyzing data from a variety of sources. In one aspect, the techniques are directed to generating a summary prior to the encounter by curating data from a variety of sources and analyzing temporal data to determine temporal patterns and/or other factors within the data that are relevant to at least one symptom that was a reason for the encounter. In another aspect, the techniques are directed to obtaining an audio including a conversation during the encounter and updating the summary (e.g., in real time) based on analyzing the audio and, in some examples, obtaining additional data based on the analysis.

### TECHNICAL FIELD

The present disclosure generally relates to techniques for processing encounter-related information. More specifically, the disclosure relates to systems and methods for generating a concise and/or dynamic digest or summary of individual-specific information related to a reason for an encounter, by accurately and efficiently curating and analyzing data from one or more sources.

### BACKGROUND

In preparation for an encounter with an individual seeking a service (e.g., an encounter between a patient and healthcare provider), a provider typically reviews/examines information pertaining to the individual. In particular, the provider may search this information to attempt to identify information relevant to the reason for the encounter, such as a symptom that caused the individual to seek the medical service. The proliferation of data sources that may provide such information (e.g., wearable healthcare devices, Internet of medical things (IoMT) devices, electronic records of the individual's activities, etc.) offer an ever-increasing amount of potentially relevant data. However, the sheer volume and variety of such data can pose a great challenge to the provider who must distill/understand the pertinent information prior to the encounter. Moreover, during the encounter, a provider typically obtains additional information from the individual through a conversation (e.g., a patient-doctor dialog). Efficiently and accurately integrating such information into the individual's record poses yet another challenge.

### BRIEF DESCRIPTION OF THE DRAWINGS

FIG. 1 depicts an example computing environment in which various embodiments and aspects of the present disclosure can be implemented.

FIG. 2 depicts an example summary relevant to a symptom that was the reason for an encounter between an individual and the provider generated using the techniques of the present disclosure.

FIG. 3 depicts a flow diagram of an example computer implemented method, in accordance with an aspect and its various embodiments described in the present disclosure.

FIG. 4 depicts a flow diagram of an example computer implemented method, in accordance with another aspect and its various embodiments described in the present disclosure.

### DETAILED DESCRIPTION

Broadly speaking, the present disclosure relates to techniques (e.g., hardware, software, machine-learned model(s), or a combination thereof; process(es)) for accurately and efficiently curating data from a variety of sources for an encounter between an individual and a provider (e.g., a provider of medical services). Some of the disclosed techniques relate to generating a digest or summary (referred to herein primarily as a “summary”) prior to the encounter, while others relate to updating such a summary during the encounter. More specifically, the disclosed techniques include computer-based systems and processes for determining a reason for an encounter between an individual and a provider, aggregating information of potential relevance to the reason for the encounter, and presenting information in a provider-friendly format. Further still, certain disclosed techniques include obtaining and processing provider input to augment algorithmic evaluations of encounter relevance.

The disclosed techniques can take advantage of ever-proliferating sources of information that may be relevant to an encounter between an individual and a provider. The individual may be a person experiencing health-related symptoms, and the provider may be a doctor or other medical professional with whom the individual has a scheduled appointment, for example. In traditional approaches, the provider may need to manually select information of relevance to the encounter (e.g., based on symptoms, patient history, etc.) from presented information that may include irrelevant information and/or omit some information of relevance.

While it is possible to automate the curation of relevant information, it is challenging to do so in an accurate and efficient manner. Various techniques of the present disclosure solve this technical problem in a number of ways. In a first aspect, the disclosed techniques include obtaining a particular class of data associated with an individual, sensor data, and analyzing the sensor data to establish relevance to the symptoms and/or conditions associated with the encounter. These techniques may generate a data set that includes temporal patterns within the sensor data and analyze the temporal patterns for relevance to the encounter (i.e., to the symptoms and/or conditions associated with the encounter). The disclosed techniques of the first aspect may further include generating a summary and/or other output (e.g., text, graphics, voice) to present to the provider by way of an interface (e.g., computer display and/or speaker). The generated summary may be dynamic, e.g., with the techniques continually evaluating relevance (e.g., based on newly added data) and changing presented information accordingly during the encounter, for example.

The sensor data of the first aspect of the disclosure may include data collected by any suitable device(s), such as a personal device, a wearable or implanted medical device, an environmental monitoring sensor, and so on. The disclosed techniques may determine one or more temporal patterns within the sensor data (e.g., baseline data and/or variations with respect to a baseline) and determine whether the temporal pattern(s) is/are relevant to the encounter. The disclosed techniques may also determine relevance to the encounter based on other data, such as data indicative of one or more symptoms associated with the encounter, a medical condition or diagnosis (e.g., in the case of a follow-up encounter), and/or a previous determination of relevance, for example. Furthermore, the disclosed techniques may generate output text indicative of the temporal pattern relevant to the encounter.

Additionally, the techniques may generate a data set that includes the data indicative of the temporal pattern. The disclosed techniques may also add to the generated data set additional data that may or may not be generated by sensors. That is, the disclosed techniques may aggregate data from multiple sources into a common data set. Regardless of the number of sources, such a data set is referred to in this disclosure as “personal relevant information” or “PRI”. The disclosed techniques may generate an initial version of PRI prior to the encounter and then update the PRI during the encounter. For example, the disclosed techniques may add an audio stream of the encounter (i.e., an audio stream capturing the conversation between the individual and the provider), a text transcription of the audio stream, one or more outputs of an analysis of the audio stream, provider notes, etc., to the PRI. Furthermore, the techniques may update PRI with sensor data and/or any other suitable information that is missing prior to the encounter.

The techniques of this disclosure may generate, based on the PRI, a summary (which may be referred to as a patient summary, a report, a patient report, and/or other suitable term) of relevant information. That is, the techniques may add generated output text (possibly including symbols or icons) indicative of the temporal pattern relevant to the encounter to the summary. The techniques may provide the output/summary to the provider as a text display, or convert the text to audio, for example.

In some aspects, the techniques of this disclosure include two or more determinations of relevance. For example, the techniques may use a first determination of relevance to determine what data is included in PRI, and a second determination of relevance to determine what portion of the data in PRI is suitable for presentation within the summary. That is, the disclosed techniques may make the second determination of relevance to parse the PRI data set and select and format PRI data for presentation in the summary. The selection and formatting of the summary may be a dynamic process. That is the summary presented to the provider may (e.g., in real-time during the encounter) add data from PRI and/or drop data from the summary, as described in more detail below.

The techniques of this disclosure may generate, use, and/or update machine learned models for determining relevance. The techniques of this disclosure may update such models based on provider feedback. In some examples, a machine learned model may be personalized to a particular provider.

The techniques of this disclosure may be implemented within a computing environment that includes cloud computing and/or local servers communicatively connected to terminal computing devices (e.g., monitors, desktop or notebook computers, and/or personal computing devices such as smart phones and tablets).

The disclosed techniques can provide various technical advantages over other possible automation techniques. For example, the techniques efficiently and accurately generate a set of information relevant to an encounter between an individual and a provider. Furthermore, the techniques efficiently and accurately generate a summary for the provider and update the summary during the encounter.

At least in part, such advantages stem from analyzing temporal data (e.g., a collection of raw sensor readings that were generated at different times) to determine temporal patterns relevant to one or more symptoms that gave rise to an encounter (e.g., symptom(s) of a condition that causes a patient to visit a medical professional). For example, for a particular set of raw temporal data, the techniques may determine a baseline, a deviation from the baseline, and relevance to the encounter in view of temporal correlation between the deviation and a symptom.

In comparison to merely obtaining temporal data and presenting that raw data in a summary, the determining of temporal patterns can improve the relevance, accuracy, and/or precision of the summary, and the efficiency of generating such a summary. For example, while it may generally be unclear whether a certain portion of raw temporal data is relevant to a symptom, identifying a pattern within that temporal data that correlates to the symptom (e.g., temporally correlates to the system) can confirm the relevance in a highly accurate manner. Therefore, the text generated by the described techniques may more accurately present relevant data, thereby obviating additional processing and/or analysis and improving the efficiency and/or the outcome of the encounter. Moreover, identifying patterns within temporal data that correlate to the symptom can facilitate the generation of a more concise summary that excludes distracting, less relevant information.

Thus, curating a data set with temporal data, and identifying temporal patterns based on such data, enables the efficient generation of an accurate, relevant, and concise summary. The process can also advantageously enable dynamic revisions to the summary without the need to communicate with the servers and/or other sources from which the original data was obtained and/or curated, in some embodiments. In this manner, curating potentially relevant data, and performing analysis to determine temporal patterns and their relevance based on the curated data, can further improve efficiency of the system.

A second aspect of the techniques described in this disclosure includes obtaining an audio stream during an encounter between an individual and a provider (e.g., recording a patient-doctor dialog) and analyzing the audio stream (e.g., using natural language processing (NLP) techniques). Based on the analysis, the techniques may determine one or more objects of speech (e.g., term, phrase, concept, qualifier, command, etc.). Analyzing the speech can improve the accuracy of a summary for the provider, as described below.

The determined object of speech may point to relevance of a data set associated with the individual, and based on the determined objects of speech, the techniques may access the data set and determine one or more factors (e.g., sensor data, baseline data, demographic data, event data, prescription data, symptoms, etc.) associated with the individual. The techniques may then update a summary that is to be, or is currently being, presented to a provider based on the analysis of the audio stream and the determined one or more factors. Furthermore, the techniques may update PRI (e.g., as discussed above) based on the analysis. Dynamically updating the summary presented to the provider based on determined objects of speech may therefore improve the accuracy of the summary. In embodiments where this second aspect of the disclosure is combined with the above-noted first aspect (e.g., in which a summary is generated prior to the encounter), an efficiency improvement can stem from the fact that the techniques update/operate on an already-existing summary, rather than generating new and possibly redundant information, and leveraging the ready access of data sets that were curated prior to the encounter. Combining generating the summary prior to the encounter and updating of the summary during the encounter, e.g., by processing an audio stream of a conversation, can obviate the need to use separate systems for summary generation and processing the conversation, and streamlines the medical encounter.

Furthermore, the disclosed techniques may use the determined objects of speech, the associated relevance factors, and/or other associated determined information to update a machine-learned model for evaluating relevance. Updating the machine learned model based on processing of the audio stream during the encounter can improve the future accuracy of curating information relevant to the encounter.

It should be appreciated that the advantages and technical improvements described above and elsewhere herein are not the only advantages and/or technical improvements that may be realized as a result of the techniques described herein. Other advantages and/or technical improvements to the functioning of a computer itself or other technologies or technical fields may be apparent to one of ordinary skill in the art. Moreover, the techniques described herein may be readily applied in any suitable field for any suitable purpose.

### Example Computing Environment

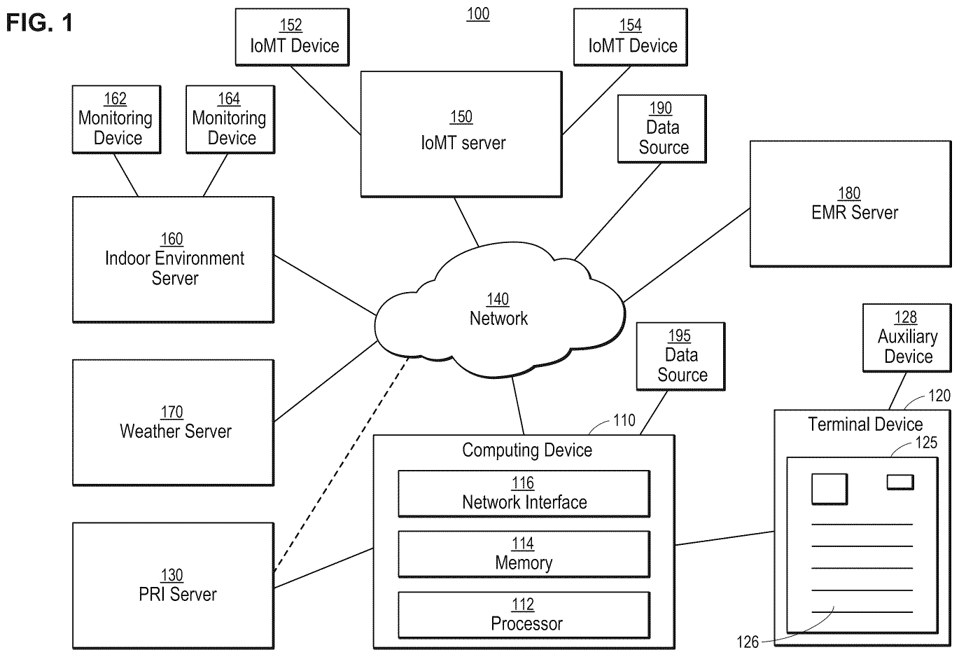

FIG. 1 depicts an example computing environment 100 in which various embodiments of the present disclosure may be implemented. In some embodiments, the example computing environment 100 generates and/or updates text relevant to an encounter between an individual (e.g., a patient) and a provider (e.g., a provider of medical services, a physician, a physician's assistant, a nurse, a counselor, etc.). Generally, the example computing environment 100 includes a computing device 110, the computing device including a processor 112, a memory 114, and a networking interface 116. The computing device 110 is communicatively connected to a terminal device 120. The terminal device 120 may include, for example, a laptop computer and/or other personal computing device (e.g., a smart phone, a tablet, etc.). The terminal device 120 may be configured to render on an output device or component (e.g., a monitor, touchscreen, etc.) a graphical user interface (GUI) 125 to display the text (e.g., text 126 discussed below) generated by the computing environment 100. The GUI 125 may also display icons and/or symbols interpretable by the provider. The text and/or icons/symbols may be a portion of a larger summary that is relevant to the encounter and rendered in the GUI 125.

In the example illustrated in FIG. 1, the terminal device 120 is in communicative connection with an auxiliary device 128. In some examples the auxiliary device 128 may be an input/output device (e.g., speaker, microphone, keyboard, mouse, trackpad, joystick, etc.) configured to facilitate control of the terminal device 120 by a user (e.g., a practitioner). In other examples, the auxiliary device 128 is a data source. The auxiliary device 128 may include a medical or personal computing device of an individual, a microphone configured to record an audio stream during an encounter, a camera configured to generate images relevant to an encounter, etc.

The computing device 110 is communicatively connected to a personal relevant information (PRI) data set server 130. In some such example computing systems, the PRI data set may be stored within the memory 114 of the computing device 110, and/or the PRI server 130 may be integrated with the computing device 110. In the example of FIG. 1, the computing device 110 may use the processor 112 to generate and/or update the PRI data set stored at the PRI server 130. The computing device 110 may be communicatively connected to the PRI server 130 via the network 140.

In some embodiments, the network 140 is communicatively connected to an Internet of medical things (IoMT) server 150. The IoMT server 150 is communicatively connected to IoMT devices 152 and 154 (or, in other examples, more or fewer IoMT devices). The IoMT devices 152 and 154 may include sensors associated with one or more individuals, and may include implantable, wearable, and/or external devices. The IoMT devices 152 and 154 may include fitness trackers, heart rate monitors, respiration monitors, temperature sensors, infusion or insulin pumps, continuous glucose monitors (see GM's), wireless vital sign monitors, portable blood gas analyzers, weight scales, continuous positive air pressure (CPAP) machines, pacemakers, security cameras, smart belts, smart rings, smart water bottles, etc. The IoMT devices 152 and 154 may collect health parameters including, for example, blood pressure, body temperature, skin temperature, heart rate variability, heart rate, resting heart rate, breathing rate, blood glucose, oxygen saturation, stress levels, electrocardiograms (ECGs), and/or other suitable parameters. With reference to the IoMT devices 152 and 154, as well as other devices of this disclosure, collecting parameters may include generating data pertaining to the said parameters. The IoMT devices 152 and 154 may collect sleep patterns, for example, by monitoring respiration, movement, and/or brain wave activity. The IoMT devices 152 and 154 may collect movement data associated with an individual, such as number of steps, stairs, movement speed, gait information, posture, etc.

The computing device 110 may collect data generated by the IoMT devices 152 and 154 from the IoMT server 150 via the network 140. In some examples, the computing device 110 may collect data from an IoMT device and/or any other suitable sensor/device via a direct wired or wireless communicative interface, i.e., bypassing the network 140.

The network 140 is communicatively connected to an indoor environment server 160. The indoor environment server 160 is communicatively connected to indoor environment monitoring devices 162 and 164 (or in other examples, to more or fewer indoor environment monitoring devices). The indoor environment monitoring devices 162 and 164 may include smart home devices, such as thermostats, air conditioners, dust detectors, air purifiers, etc. The indoor environment monitoring devices 162 and 164 may collect indoor environment parameters including visible light brightness, ultraviolet (UV) irradiation, particulate and/or chemical concentrations, water contaminants, temperature, humidity, sound volume, etc.

The computing device 110 may collect data generated by the indoor environment monitoring devices 162 and 164 from the indoor environment server 160 via the network 140. In some examples, the computing device 110 may collect data from an indoor environment monitoring device and/or any other suitable sensor via a direct wired or wireless communicative interface, i.e., bypassing the network 140.

The network 140 may be communicatively connected to a weather server 170. The weather server 170 may collect and store weather data, including temperature, pressure, precipitation, wind speed, pollen counts, UV irradiation, etc., for a variety of geographic locations. The computing device 110 may collect data from the weather server 170 by way of the network 140.

The network 140 may be communicatively connected to an electronic medical records (EMR) server 180. The computing device 110 may collect EMR data associated with an individual via the network 140. In some examples, the EMR server 180 may be on a local area network (LAN) and/or another communicative connection with the computing device 110, that bypasses the network 140. The EMR server 180 may include a variety of medical information (e.g., medical history, medical test results, reports from previous encounters, etc.) associated with an individual.

In FIG. 1, the network 140 is communicatively connected to an additional data source 190 which may be a server, an individual device, etc. For example, an IoMT device may connect by way of the network 140 to the computing device 110 without first connecting to the IoMT server 150. The network 140 may be communicatively connected to a variety of other data sources (e.g., servers, databases, individual devices, etc.). The computing device 110 may have access to the variety of data sources connected to the network 140. For example, the computing device 110 may have access to geographic location history, a calendar, nutrition, electronic device usage, screen time, and/or browsing history of an individual, based on the respective permissions (e.g., opt-in by the individual). To that end, the computing device may be connected (e.g., directly or by way of the network 140) one or more personal electronic devices of the individual and/or servers.

The computing device 110 is communicatively connected to a local data source 195 via a direct communicative connection, bypassing the network 140. The local data source 195 may be an IoMT device, a personal electronic device, an office recording system, etc. For example, an individual may bring the local data source 195 to the encounter and connect the data source to the computing device 110 (e.g., a fitness tracker, a smart phone, and attached or implanted medical device, etc.) to upload data to the computing device 110. In some examples, the local data source 195 may be a sensor monitoring an encounter between an individual and the provider. For example, the local data source 195 may include a microphone for recording an audio stream during the encounter. Additionally or alternatively, the data source may include a camera that captures sign language, gestures, and/or expressions associated with the encounter for later or real-time interpretation by computing device 110. Furthermore, the computing device 110 may have access (via the network 140 and/or a direct connection) to one or more knowledge bases (e.g., proprietary knowledge bases, medical reports, medical journals, etc.).

In operation, the computing device 110 may access data associated with an individual associated with an encounter from the IoMT server 150, the indoor environment server 160, the weather server 170, the EMR server 180, the additional data source 190, and/or the local data source 195. The computing device 110 may be communicatively connected to and access data from a variety of other servers and/or devices generating and/or storing data associated with an individual associated with an encounter. Furthermore, the computing device 110 may access data from the terminal device 120 and over the auxiliary device 128 connected to the terminal device 120.

The computing device 110 may process the accessed data using one or more processors within the processor 112 by executing instructions stored in the memory 114. Additionally or alternatively, the computing device 110 may use one or more remote processors (e.g., within a cloud computing environment), for example, by accessing an application programming interface (API). The computing device 110 may process the data using a machine-learned model stored locally in the memory 114 or as a cloud computing resource accessible through the network 140. The machine-learned model may be trained using the computing device 110, another computing device, and/or a combination thereof. The computing device 110 may generate text and/or other suitable output to be presented to a provider by way of the terminal device 120 based on the analysis of the data.

It should be noted that FIG. 1 is but one example computing environment 100 and the techniques of the disclosure may be practiced using other computing environments. In alternative computing environments, for example, at least a portion of the computing device 110 may be disposed on the cloud. Additionally or alternatively, the PRI server 130 may be a cloud resource. In other examples, the PRI server 130 may be replaced by a database stored within the memory 114 of the computing device 110. In some examples, the PRI data may be distributed and/or replicated or mirrored for speed and efficiency of executing the techniques of the disclosure.

It should be appreciated that, while the computing device 110, the terminal device 120, the PRI server 130, network 140 are illustrated in FIG. 1 as single components, the example computing environment 100 may include multiple (e.g., tens, hundreds, thousands) of computing devices, terminal devices, PRI servers, and/or networks. Furthermore, the example computing environment 100 may include additional multiple (e.g., tens, hundreds, thousands) servers and/or additional multiple (e.g., tens, hundreds, thousands) data-generating devices.

The processor 112 may include any suitable number of processors and/or processor types. In some examples, the processor 112 includes one or more central processing units (CPUs), one or more graphics processing units (GPUs), one or more tensor processing units (TPUs), one or more field-programmable gate arrays (FPGAs), one or more application-specific integrated circuits (ASICs), and/or the like. Generally, the processor 112 comprises hardware configured to execute code/instructions stored in the memory 114.

The networking interface 116 may comprise one or more hardware components to generally enable the computing device 110 to communicate via one or more network(s) (e.g., network 140) with other components and/or devices of the computing environment 100, such as the servers 130-180, the computing device 110 itself (e.g., between components of the computing device 110), and/or other suitable devices or combinations thereof. More specifically, the networking interface 116 may enable the computing device 110 to communicate with any component of the example computing environment 100 across the network 140. The networking interface 116 may comprise hardware and/or software that operates according to at least one communication protocol of the network 140.

The network 140 may include wired and/or wireless communication network(s) such as a cellular network (e.g., 5G®, 4G LTE®, 3G®), a Wi-Fi® network (802.11 standards), a microwave access network (e.g., WiMAX®), and/or any other suitable wide area network (WAN), local area network (LAN), personal area network (PAN), etc. Moreover, the network 140 may be a single communication network, or may include multiple communication networks of one or more types (e.g., one or more wired and/or PANs or LANs, and/or one or more WANs such as the Internet). In some embodiments, the network 140 includes multiple, distinct networks (e.g., one or more networks for communications between servers 130-180 and computing device 110, and a separate, Bluetooth® or wireless LAN (WLAN) network for communications between the computing device 110 and the terminal device 120 and/or the PRI server 130, and so on).

The memory 114 may include any suitable memory type(s), including one or more volatile memories (e.g., dynamic and/or static random-access memory (RAM)) and/or non-volatile memories (e.g., read-only memory (ROM), erasable programmable ROM (EPROM), electrically EROM (EEROM), NAND flash, and/or solid state drive(s) (SSD(s))), all or any of which are examples of non-transitory computer-readable media. In some examples, the memory 114 stores one or more of: an operating system; one or more software components (e.g., firmware, application(s), binary, source code, executable instructions, machine-learned model(s)); transient data and/or code loaded and/or operated on by one or more software component(s); and/or other suitable components/data. In some examples, the memory 114 stores an application for generating text for the encounter based on received data, and application for updating text for the encounter based on a recorded audio stream, and/or other suitable applications for executing the techniques of this disclosure. The memory 114 may additionally or alternatively store one or more databases.

As discussed further below, the memory 114 may store a machine-learned model, which may comprise generative machine-learned model component(s), such as a transformer-based machine-learned model (e.g., a large-language model (LLM), an embedding model, a diffusion model, and/or the like); and may additionally or alternatively comprise other machine-learned model component(s), such as neural network(s), decision tree(s), and/or the like. In some examples, the machine-learned model may be trained to use text as input and may generate text or, in other embodiments, may be a multimodal LLM that operates upon and/or generates text and also other types of content (e.g., text, images, audio, etc.). The machine-learned model may receive a text prompt (referred to herein at times as simply a “prompt”) as an input, process the text prompt, and output text content responsive to the text prompt. The machine-learned model may additionally or alternatively include a deep neural network and may perform various natural language processing tasks to understand a text query/prompt and generate a response to the text query/prompt, e.g., as part of a pre-processing operation and/or a post-processing operation. For example, in a pre-processing operation a neural network and/or another transformer-based machine-learned model may be trained to augment the original prompt to add sufficient context, determined to be associated with the prompt or the encounter. In a post-processing operation, the neural network and/or another transformer-based machine-learned model may be trained to review and alter (e.g., as necessary or on any other suitable time basis) an output of a transformer-based machine-learned model. For example, this review and alteration may comprise altering text generated by a first transformer-based machine-learned model to correct errors, translate the text, translate speech to text and/or text to speech, and/or the like.

The machine-learned model may have a transformer-based model architecture that comprises an encoder that tokenizes the input and determines embeddings for the tokens, and a decoder that generates the output based at least in part on the embeddings. The transformer model may incorporate self-attention and/or cross-attention mechanisms to facilitate more accurate output. In some embodiments, such a transformer-based machine-learned model may include different configurations of self- and/or cross-attention, followed by neural network(s) (e.g., feedforward layer(s)), recurrent layer(s), aggregation layer(s) (e.g., using softmax, matrix multiplication, and/or other aggregation techniques), and/or the like. The machine-learned model may be a general-purpose model (e.g., trained on a wide array of publicly available datasets such as web pages, documents, etc., available via the Internet) such as generative pre-trained transformer (GPT) 3.5, bi-directional encoder representations from transformers (BERT), or a domain-specific model (e.g., trained and/or fine-tuned on custom and/or proprietary datasets).

In some examples, the functionality described herein for computing device 110, the terminal device 120, and/or the PRI server 130 is collectively performed by a single computer (e.g., desktop computer, laptop computer, terminal), a mobile device, a wearable, augmented reality glasses/headsets, virtual reality glasses/headsets, mixed or extended reality glasses/headsets, and/or other suitable computing device.

### Example Generated Summary

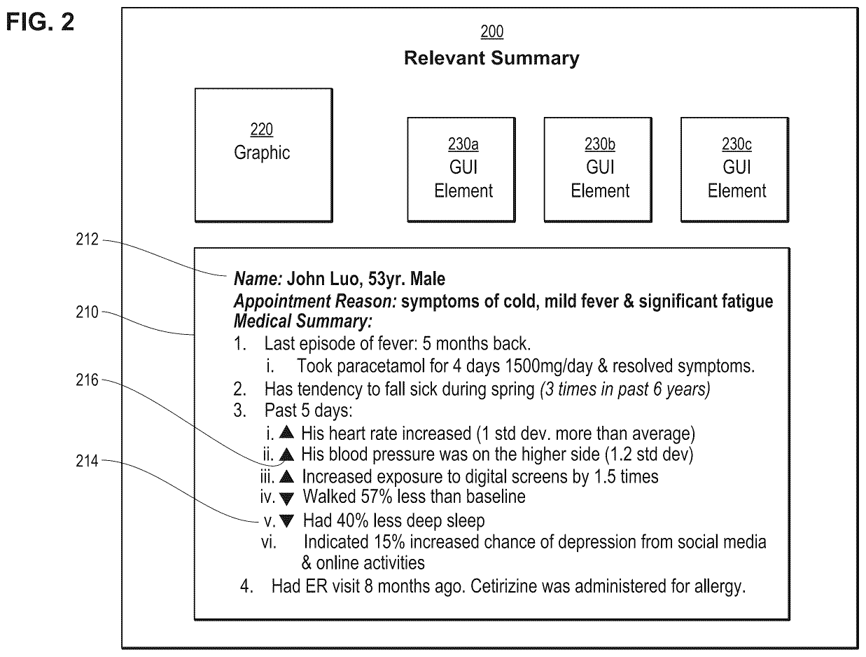

FIG. 2 illustrates an example summary 200 (e.g., text 126) generated for an encounter between an individual and a provider, which may be displayed via a GUI rendered on a terminal device (e.g., terminal device 120), using the techniques of this disclosure. The example summary 200 includes a text portion 210 which includes a reason 212 (e.g., one or more symptoms, and possibly a medical condition, etc.) for the encounter (e.g., appointment). Furthermore, the example summary 200 includes example output text that may be indicative of a temporal pattern. The output text shown in FIG. 2 is but one example of the texts in the example summary 200. Certain texts within the example summary 200 are labeled and/or enumerated, and/or include symbols (e.g., symbol 216) that may be indicative of temporal patterns. With respect to the latter, for example, the symbol 216 is an upward pointing triangle indicating a temporal pattern that corresponds to an increase of a value over time. Other symbols include other upward pointing triangles, as well as downward pointing triangles indicative of temporal patterns that correspond to a decrease of a value over time. While triangle icons are illustrated in the example summary 200, arrows or any other suitable symbols may illustrate information pertinent to the temporal patterns and/or any other relevant information.

In the example summary 200, the texts, which may be referred to herein as text “items” (e.g., text item 214), may include indications of temporal patterns such as increased heart rate, elevated blood pressure, increased exposure to digital screens, decreased walking, decreased quality of sleep, etc. Furthermore, the example summary 200 includes text items indicative of a previous episode of the symptom, and indication of a chance of depression based on online activities, and a previous emergency room visit. In other examples, text items may include temporal patterns and/or any other relevant information based, for example, on the data sources described with reference to FIG. 1.

To generate output text (e.g., text item 214) in the summary 200, the computing device 110 may obtain data indicating a reason (e.g., reason 212) for an encounter between an individual and the provider. The computing device 110 may further obtain sensor data generated by one or more devices associated with the individual (e.g., devices 152, 154, 162, and/or 164). The computing device 110 may generate a data set comprising data indicative of one or more temporal patterns in the obtained data. The generated data set may be stored at the PRI server 130. The computing device 110 may determine one or more temporal patterns based on the data set and determine that the temporal patters are relevant to the encounter. Each of one, some, or all of the temporal patterns described in the text items (e.g., text item 214) within the summary 200, along with accompanying icons (e.g., symbol 216), is relevant to at least one of the symptoms within the reason 212 for the encounter.

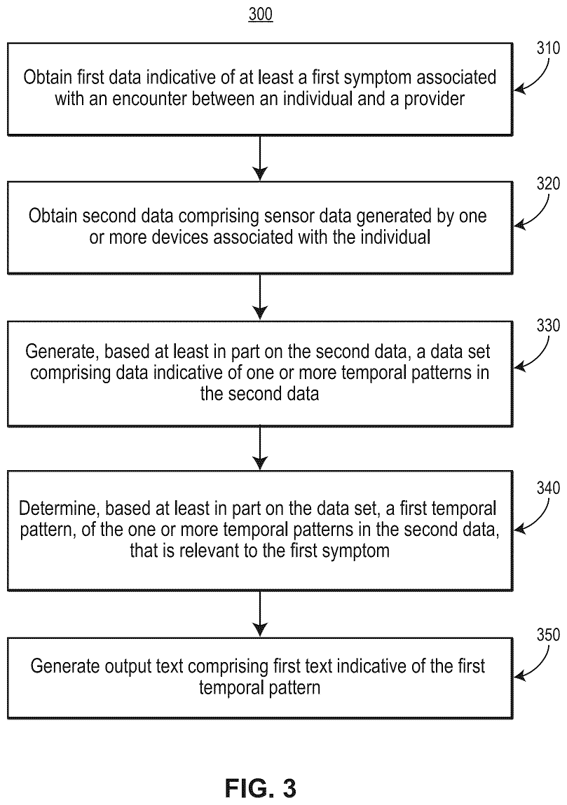

Besides the text portion 210, the example summary 200 includes a graphic 220. The graphic 220, for example, may be a photograph of the individual. In other examples, the graphic 220 may include a graphical representation of a temporal pattern. Furthermore, the summary 200 includes GUI elements 230a-c which may be integrated into a GUI within which the summary 200 is displayed (e.g., rendered on a terminal device 120). Example functionalities of the GUI elements 230a-c are described below with reference to a dynamic property that the summary 200 may have in some embodiments. An example method for generating the summary 200 and, particularly, for generating text elements (e.g., text item 214) within the summary 200 is discussed in more detail with reference to FIG. 3.

As mentioned above, the summary 200 can be dynamic, e.g., configured to change during an encounter between an individual and a provider. The changes to the summary 200 may be based, for example, on an audio stream indicative of a conversation between the individual and the provider. One of the elements 230a-c may be a virtual button to toggle between revising a summary 200 and initiating dynamic aspects of the summary 200. One of the elements 230a-c may be a virtual button to toggle between initiating/continuing and stopping recording and/or processing an audio stream. One of the elements 230a-c may be a virtual button to initiate and upload of additional sensor data or other relevant information to be stored (e.g., at the PRI server 130) and/or used to update the summary 200.

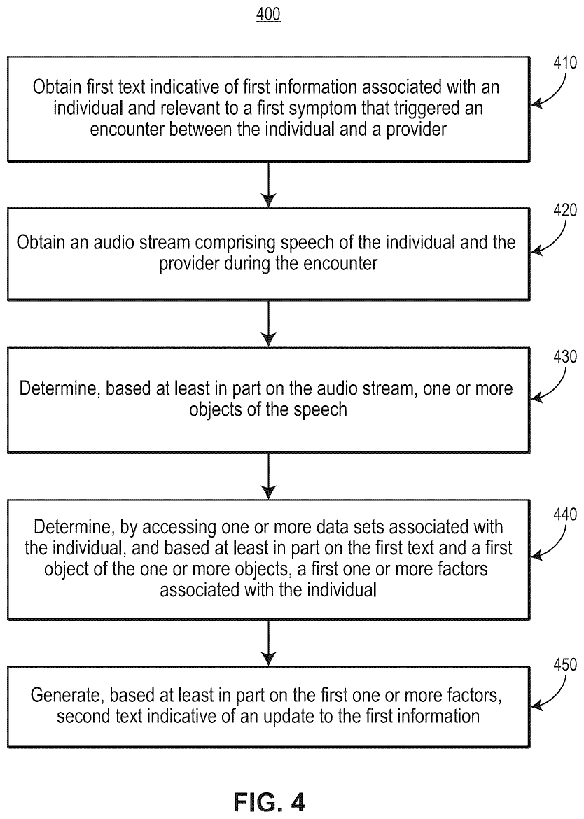

To dynamically change the summary 200, the computing device 110 may obtain/analyze one or more text elements (e.g., text item 214) indicative of information associated with an individual and relevant to a first symptom (e.g., within the reason 212) that triggered an encounter between the individual and the provider. The computing device 110 may also obtain an audio stream comprising speech of the individual and the provider during the encounter and determining one or more objects of the speech (e.g., concepts, terms, phrases, qualifiers, commands, etc.). Obtaining and/or analyzing the audio stream may include using the local data source 195 (e.g., a microphone, a recording system, etc.), and may occur in response to the provider pressing a virtual button (e.g., one of elements 230a-c). The computing device 110 may further determine, based at least in part on the text element and the object of speech, an update to information described in the text. For example, the text item 214 may include information about a reduction in deep sleep, and the conversation between the individual and the provider may include the individual mentioning that they lent their sleep tracker to another person. Consequently, the computing device 110 may downgrade the relevance of the sleep data and remove the text item 214 from the summary. In another example, the individual may discuss going on a cycling trip during the previous five days, and the determined objects of speech may include the cycling trip and/or the related discussion between the individual and the provider. The determined objects of speech, when analyzed by the computing device 110, may offer context to the text items pertaining to increased heart rate and decreased walking. The text items may change accordingly to add such context. Additionally or alternatively, the computing device 110 may add new text to the summary without necessarily revising or removing existing text. An example method for updating the summary 200 and, particularly, for updating text elements (e.g., text item 214) within the summary 200 is discussed in more detail with reference to FIG. 4.

It should be noted, that the relative size and position of the elements 210, 220, 230a-c, etc., may change in different examples. Furthermore, the summary 200 may include a variety of other suitable elements. Some of the elements may be static, some of the elements may be dynamic, and some of the elements may be inputs within a GUI.

### Example Computer-Implemented Methods

FIG. 3 depicts a flow diagram of an example computer-implemented method 300 for generating a summary (e.g., summary 200) and, particularly, for generating one or more text elements within the summary, in accordance with various embodiments described in the present disclosure. The method 300 may be implemented by any suitable system, such as one or more components of FIG. 1 (e.g., computing device 110, or more specifically processor 112, and possibly also one or more other components of FIG. 1).

At block 310 the system implementing the method 300 obtains first data indicative of at least a first symptom associated with an encounter between an individual and a provider. For example, an individual may sign up for an appointment/visit (i.e., an encounter) with a provider using the provider's appointment system. The appointment system may prompt the individual to enter a reason for the appointment. The system implementing the method 300 may add the reason for the appointment to a PRI data set, or generate a new PRI data set. In some examples, the PRI data set is a new data set generated specifically for the appointment. In some examples the PRI data set may represent multiple encounters between the individual and one or more providers. The system implementing in the method 300 may generate data indicative of one or more symptoms entered by the individual, such as a code, standard terms for the one or more symptoms, etc. Furthermore, the appointment system may prompt the individual to enter additional information, for example, indicative of medical conditions, demographic information, etc. The system implementing in the method 300 may obtain their additional information from the appointment system. Additionally or alternatively, the system may obtain additional information from the electronic medical records associated with the individual. The system may use the additional information along with the symptoms to compute relevance, as discussed further below.

In some embodiments, the system implementing the method 300 may obtain reason codes (e.g., as part of the first data) or standard descriptors of the reason for the encounter based at least in part on the information provided by the individual or another responsible party (e.g., a parent, a guardian, a referring medical professional, etc.). In some examples, the provided information (and/or other first data) may include unstructured data (e.g., text, images, audio, etc.). The system may use natural language processing (NLP), natural language understanding (NLU), automatic speech recognition (ASR), optical character recognition (OCR), computer vision, and/or any other suitable computing technique to process the unstructured data for obtaining the reason for the encounter (e.g., a reason code, a symptom, etc.). The reason code may be a descriptor for a symptom such as “sleep problem,” “stomach upset,” “stomachache,” “rash,” etc., and/or an associated alphanumeric code, for example.

At block 320 the system implementing the method 300 obtains second data comprising sensor data generated by one or more devices associated with the individual. The system implementing the method 300 may obtain data from one or more sensors (e.g., devices 152, 154, 162 and 164) as discussed with reference to FIG. 1, for example. The second data may include time series data. The system may obtain time series data for a determined time interval (e.g., 1, 2, 5, 10, 20, 50, or any other suitable number of days). The sensors generating the time series data may be IoMT sensors, such as sensors of IoMT device 152 and/or 154. For example, a fitness tracker or another suitable device may provide data indicating fitness routines and/or exercise regiments as well as general activity levels. The system may determine the duration of the time interval for which to collect the data based at least in part on the symptoms for the encounter, for example (e.g., based on possible causes for the symptoms and previously established associations between the possible causes and the temporal pattern in data). In one specific example, for a certain possible infection that causes a rash, the rash may be preceded by a rise in body temperature in the previous several weeks. Accordingly, the system may collect sensor data indicative of body temperature for several weeks prior to the onset of the rash. In another example, when a known potential cause of a symptom is glucose dysregulation, the system may collect a suitable duration (e.g., the previous 1 week, 2 weeks, 3 weeks, etc.) of sensor data associated with glucose monitoring and/or insulin injection data.

The second data obtained at block 320 may include any suitable sensor data possibly pertaining to the reason for the encounter. For example, the second data may include sensor data from non-medical internet of things (IoT) devices such as smart home devices (e.g., thermostats, air conditioners, dust detectors, air purifiers, light controllers and/or detectors, speakers, etc.). The smart home devices may include indoor environment monitoring devices 162 and/or 164 of FIG. 1, for example. The second data may include sensor data from the individual's residence, workplace, and/or any other suitable environment where the individual spends time. The second data may include data on air quality, temperature, humidity, noise levels, illumination levels, and so on. The second data may be obtained via a network (e.g., network 140) from a server (e.g, indoor environment server 160). Additionally or alternatively, the system implementing the method 300 may obtain at least some of the second data from a personal device of the individual. For example, the personal device may download sensor data from an indoor environment sensor via a Bluetooth or Wi-Fi connection and, subsequently, upload the data via a direct connection to a computing device (e.g., computing device 110) of the system implementing the method 300. In some examples, the second data may include weather data. For example, the system may obtain temperature data, humidity data, precipitation data, daylight brightness data, etc. from a weather server (e.g., weather server 170).

The second data at block 320 may include data other than sensor data. For example, the second data may include digital device usage data (e.g., screen time, application usage, social media usage, and/or search history). Additionally or alternatively, the second data may include digital calendar data of the individual (e.g., appointments, work schedule, travel, etc.). Additionally or alternatively, the second data may include nutrition information (e.g., meal frequency and/or timing, food consumed, caloric intake, caffeine use, vitamin intake, etc.). The nutrition information may be recorded by the individual in written, spoken, and/or image/video formats. Additionally or alternatively, the second data may include application data generated by an application installed on a mobile device of the individual and configured to receive manual entries of activities. Such data may be indicative of activities during a time period. Additionally or alternatively, the second data may include any medical data (e.g., from EMR server 180) of potential relevance. The medical data may include previous encounter data (e.g., dates, reasons, symptoms, prescriptions, diagnoses, etc.), medication data, test results, etc.

Temporal patterns in sensor data, baseline data, demographic data, event (travel, previous appointments, etc.) data, prescription data, symptoms, and/or other data or information associated with an individual is generally referred to herein as “factors” relating to that individual. At blocks 310 and 320, the system implementing the method 300 may obtain a variety of factors associated with the individual.

At block 330 the system implementing the method 300 generates, based at least in part on the second data, a data set comprising data indicative of one or more temporal patterns in the second data. The data set may be a PRI data set stored and maintained by PRI server 130, for example. As used herein, “generating” data (or a data set, etc.) can encompass generating entirely new data, or modifying existing data. In some embodiments, the data set generated at block 330 includes all collected time series data of potential relevance to the encounter. That is, the system may add time series data to the data set regardless of variations in parameter values in the time series data. In other embodiments, the system may analyze the data (at block 320 or 330) to select portions of the time series data for inclusion in (and/or exclusion from) the data set. For example, the system may not store the entirety of a particular time series by omitting portions of the time series that are consistent with a baseline. The system may add information based on the values of a parameter (e.g., IoMT or environmental sensor values) to the data set when the amount of variation in the values exceeds a threshold amount. Determining the amount of variation may include determining a first mean or median value of the values of the first parameter during a particular time period, and determining a difference between the mean or median value and a baseline mean or median value for the individual. Additionally or alternatively, the system may add into the data set an indication that the respective time series was consistent with the baseline, at least during the time period. On the other hand, the system may in some embodiments determine a portion of the time series data representative of an excursion with respect to a baseline, and add only that portion to the data set.

To determine whether a portion of the time series is representative of an excursion with respect to the baseline, the system may compute and compare short- and long-term averages, variances, and/or other statistical measures within the time series. The system may use any suitable digital filters in the analysis. Additionally or alternatively, the system may use machine-learned models to determine features within the time series that are indicative of excursions with respect to a baseline. In any case, for a given time series, the system may determine to save within the data set the entire time series, a portion of the time series, and/or an indication of the excursion.

At least some of the temporal patterns stored by the system within the data set (e.g., PRI data set) may indicate specific events and associated times. For example, the system may store trips taken by the individual, environmental events (e.g., floods, dust storms, power outages, etc.), and/or any other events of potential relevance to the encounter. Generating the dataset may include obtaining data from an application (e.g., installed at the users mobile device and configured to receive manual inputs) for a specific time period. Adding at least a portion of the data set obtained from the application may include comparing the data obtained from the application during the specific time period to a baseline determined based on the data obtained from the application during another time period. For example, the data may be included and/or isolated as a temporal feature in the data set if an amount of variation exceeds a threshold amount. Determining the amount of variation may include determining a frequency of the activity to an earlier frequency of the activity.

The system may add to the data set PRI and/or EMR data associated with one or more previous encounters with similar reasons, symptoms, and/or temporal pattern combinations. In some embodiments, the system saves at least a portion of the generated data set for further reference during a potential future encounter.

Generally, the system may generate a data set of some or all factors (e.g., demographic, location, environment, biometric, nutrition, behavioral, medical, symptom, etc.) associated with the individual that the system is able to obtain. At least some of the factors may be indicative of temporal patterns. The system may restrict the data set to the factors that may be relevant based on suitable criteria. For example, factors may be included in the data set based on a time window with respect to the appointments (e.g., past decade, past year, past month, past week, etc.). The time window may depend on a particular factor. Additionally, as discussed above, the system may pre-select specific temporal features and statistical measures to store in the data set rather than storing all available time series data.

At block 340 the system performing the method 300 determines, based at least in part on the data set generated at block 330, a first temporal pattern, of the one or more temporal patterns in the second data, that is relevant to the first symptom. In some embodiments, the system may also determine, based on the data set generated at block 330, one or more additional factors (e.g., one or more additional temporal patterns, and/or other types of factors) that are relevant to the first symptom.

As discussed above, the system may determine, at least coarsely, relevance (e.g., a relevance score) of the data (e.g., temporal patterns and/or other factors) in generating the data set at block 330. Even when the system made one or more such determinations of relevance at block 330, however, the system can make one or more additional determinations of relevance at block 340. For example, if a patient visits a doctor because he or she has been suffering from fatigue, block 330 may include determining temporal patterns in sleep, screen time, nutrition, and weather conditions (because these are generally known to be potential causes of fatigue), and block 340 may include determining that, for this particular individual, the sleep and nutrition temporal patterns are more likely relevant to the individual's fatigue, while the screen time and weather condition temporal patterns are less likely relevant to the individual's fatigue. Thus, the system may at block 340 determine that both a first temporal pattern (the sleep pattern) and a second temporal pattern (the nutrition pattern) are relevant to the first symptom.

In one example, a person may seek an appointment due to the symptom of insomnia. The system implementing the method 300 may store, at block 330, data indicative of the sleep patterns, work schedule, travel schedule, device usage, stress levels, diet, prescriptions, etc. To determine relevance, at block 340, the system may determine temporal patterns (e.g., deviations from baseline) that correlate or have previously correlated with sleep disturbances. For example, when determining circadian rhythm disturbance as a possible cause, the system may determine a recent increase in international travel along with a decrease in walking during daylight hours as relevant patterns. As another example, when determining anxiety as an alternative possible cause, the system may determine elevated stress levels, changes in diet, and/or increased device usage as relevant temporal patterns.

To determine relevance, the system may access and analyze an existing knowledge base, such as medical literature, institutional reports, etc. Furthermore, the system implementing the method 300 may use previously determined dependencies and/or correlations among symptoms and a variety of temporal patterns. To that end, the system may use anonymized data from previous encounters of many individuals with providers. Furthermore, the system may generate, train, and/or use a machine-learned model to give scores to possible causes of a reason for the encounter. The system may determine relevance of a particular temporal pattern using variational analysis. More specifically, the system may deem a temporal pattern to be relevant when removing the temporal pattern from an input to the machine-learned model significantly alters scores generated by the machine-learned model.

The system may access at least portions of data sets generated for previous encounters to find similar combinations of causes, symptoms, temporal patterns, and/or other factors. For example, if a previous instance of insomnia appeared along with a prior reduction in physical activity, the system might give a higher relevance score to a subsequent reduction in physical activity accompanying the respective instance of insomnia. Generally, if a certain combination of temporal patterns previously occurred in association with a symptom, the system may give the combination a higher relevance score with respect to subsequent occurrence of the symptom. Furthermore, the system may raise the relevance score of temporal patterns and a symptom based on repeated coinciding instances among the temporal patterns and the symptom. Further still, the system may raise relevance of temporal patterns and a symptom based on recency of a prior coinciding incident between the temporal patterns and the symptom.

The system may use one or more artificial intelligence (AI), machine learning (ML), and/or deep learning (DL) techniques to evaluate relevance of a temporal pattern and/or another factor to a reason for an encounter and/or an associated symptom. The system may use regression (e.g., logistic regression, polynomial fitting, etc.), tree-based algorithms (e.g., decision trees, random forests, etc.), support vector machines (SVMs), etc. Additionally or alternatively, the system may use Shaply additive explanations (SHAP), local interpretable model-agnostic explanation (LIME), and/or any other tools suitable for interpreting AI models.

The system may use a graphical architecture to determine relevance. The nodes of the graph may represent symptoms, as well as temporal patterns and/or any other suitable factors. The edges connecting the nodes of the graph may represent relevance values. In some examples, at least some of the nodes may have associated vector representations with quantitative values for suitable descriptors (e.g., based on medical literature and/or other suitable information). The system may compute a relevance metric for an edge between two nodes based at least in part on Jaccard, cosine, and/or any other suitable similarity measures applied to respective pairs on nodes. Furthermore, the system may rank symptoms, temporal patterns, and/or other features based on the computed similarity scores with respect to the reason for the encounter. Additionally or alternatively, the similarity scores may be based at least in part on previously established correlations among factors occurring in previous encounters, medical literature, and/or any other suitable knowledge base.

In some examples, to determine that a temporal pattern and/or any other suitable factor is relevant to the encounter, the system implementing the method 300 may determine one or more candidate causes for the first symptom. The system may determine the candidate causes by accessing a knowledge base. The system may determine relevance of the temporal pattern in view of the one or more possible causes using the techniques discussed above and/or any other suitable techniques. The system may determine one or more temporal patterns and/or other factors that have comparatively high relevance to the first symptom in view of one possible candidate causes to comparatively lower relevance with respect to another possible candidate causes. The system may flag such temporal patterns and/or other factors as differential factors.

In some embodiments and/or scenarios, determining that a temporal pattern and/or any other suitable factor is relevant to the encounter is based at least in part on one or more factors linked to the symptom by a machine-learned model. For example, a temporal pattern in sleep may be linked to fatigue, which may be linked by a machine-learned model to an infection that may, in turn, be linked to confusion, which may be the first symptom. The machine-learned model may apply a graph and/or tree-based algorithm. In some examples, the system may use a machine-learned model comprising a neural network having one or more respective feature weights for the one or more factors. In some examples, when a factor may not have an associated input into a machine-learned model, another machine-learned model and/or other suitable algorithms may incorporate data related to the factor without the associated input into another factor that may have associated input. For example, activity data may be transformed in some examples into sleep pattern data. The machine-learned model may be trained (e.g., by computing device 110 or another device or computing system) using a graph. The graph may be constructed (e.g., by the system implementing the method 300) with at least some nodes representing symptoms and at least some nodes representing other factors. At least some pairs of nodes may be connected with edges associated with weights indicative of relevance and/or similarity between the nodes.

In some examples, the system may present to a provider and/or a suitable medical professional, via a GUI rendered on a display, rankings of symptoms, temporal patterns, and/or other features computed by the system. Furthermore, the system may prompt the provider and/or the suitable medical professional to evaluate and/or adjust the rankings. These adjusted rankings may serve as labeled data for training a machine learned model to determine relevance of temporal patterns, other symptoms, and/or other suitable factors to a symptom (e.g., patient complaint) that caused the encounter.

At block 350 the system implementing the method 300 generates output text comprising first text (e.g., text item 214) indicative of the first temporal pattern. In some embodiments and/or scenarios, the output text also comprises text indicative of one or more other temporal patterns that were also determined at block 340. In some embodiments, upon determining a relevance score of a temporal pattern, the system compares the relevance score to a threshold and determines that the temporal pattern is relevant if and only if the score exceeds the threshold. Additionally or alternatively, the system may rank temporal patterns according to relevance scores and determine that the temporal patterns with the highest relevance scores (e.g., the highest N scores, where N is a positive integer greater than or equal to one) are relevant to the symptom.

The generated output text may include statistical descriptors (e.g., mean, median, mode, variance, standard deviation, skew, etc.) and a corresponding time basis. The generated output text may further include indicators of direction of deviation, magnitude of deviation, frequency of deviation, and/or respective times of deviation, for example.

Generated output text may include icons or symbols, e.g., indicating a direction of a change in a temporal pattern with respect to a baseline, a temporal relationship to the onset of a symptom, a relevance score, a flag (e.g., differential factor), etc. The system may generate multiple text items in a structured or unstructured manner. For example, the text items may be organized in one or more ordered lists that are numbered, bulleted, nested, and/or indented with respect to each other. The text items may be dynamic, i.e., change order with respect to each other, be removed, edited, etc. For example, a summary including one or more text items may be updated during an encounter as discussed in more detail with reference to FIG. 4.

In some examples, the system may generate text indicative of a respective temporal pattern at block 350 using a transformer-based machine-learned model, such as a large language model (LLM) or a small language model (SLM), or a generative adversarial network (GAN), etc. In some embodiments, the system generates at least a portion of the output text in the format of a story or an essay. To that end, the system may use an LLM such as GPT-3, GPT-4, InstructGPT®, ChatGPT®, Google Gemini®, YouChat®, etc. Additionally or alternatively, the system may use a predetermined format for output text, and fill in descriptors of respective temporal patterns and/or other factors. In some examples, the system may prompt a provider for a preferred format of the output text, and apply the preferred format.

The system may render the text items on a GUI in the form of a summary relevant to a reason (e.g., the first symptom, a reason code, etc.) for the encounter. In some examples, a file representing the summary may be generated by the system. The file (or any other digital form of at least a portion of the summary) may be stored with the data set of information relevant to the encounter (e.g., the PRI data set).

FIG. 4 depicts a flow diagram of an example computer-implemented method 400 for updating a summary (e.g., summary 200) and, particularly, for updating one or more text elements within the summary, in accordance with various embodiments described in the present disclosure. The method 400 may be implemented by any suitable system, such as one or more components of FIG. 1 (e.g., computing device 110, or more specifically processor 112, and possibly also one or more other components of FIG. 1).

Generally, the method 400 is directed to monitoring a conversation between an individual and a provider during an encounter (e.g., between a patient and a doctor during an appointment). As the individual discloses new information, the system implementing the method 400 may augment the text (e.g., text items and/or icons/symbols generated by the method 300) with new, related information. To that end, a recording device (e.g., local data source 195) may record the conversation audio and pass the audio to one or more computing devices (e.g., computing device 110) that are part (or an entirety) of a system implementing the method 400. The one or more computing devices may scan the EMR and/or PRI for data that may be relevant to the encounter based on the audio of the conversation. In some examples, the method 400 may include generating (e.g., via a GUI) a prompt for the provider to confirm or reject relevance of new data to making an assessment, a diagnosis, etc. The new data may dynamically (e.g., substantially in real time) adjust a summary (e.g., a summary generated using the method 300) relevant to the encounter. In some embodiments, the device (e.g., tablet of the provider) that records the conversation audio also implements some or all of the method 400. In other embodiments, the method 400 is performed in part or entirely by a component (e.g., computing device 110) other than the recording device.

At block 410, the system implementing the method 400 obtains first text indicative of first information associated with an individual and relevant to a first symptom that triggered an encounter between the individual and a provider. The first text may include one or more text items generated at block 350 of the method 300, for example. The first text may be a part of a summary (e.g., summary 200) presented to the provider, e.g., via a GUI rendered at a display device (e.g., terminal device 120).

At block 420, the system obtains an audio stream comprising speech of the individual and the provider during the encounter. The audio stream may be obtained from the local data source 195 (e.g., via a WLAN or wired LAN), for example. In some implementations, the local data source 195 is included in the system performing the method 400 (e.g., if the system includes the terminal device 120, and if the terminal device 120 includes a microphone).

At block 430 the method 400 includes determining, based at least in part on the audio stream, one or more objects of the speech. An object of speech may be one or more phrases, a concept that is reflected by the speech but not necessarily explicitly mentioned in the speech, a confirmation or rejection of a particular fact, and so on. In some examples, determining the one or more objects of speech may include converting and/or transcribing the audio to text, and determining the object(s) using a transformer-based machine learned model. The system implementing the method 400 may convert voice to text using automatic speech recognition (ASR) and/or NLP techniques. The system may use topic modeling (e.g., bidirectional encoder representations from transformers (BERT), robustly optimized BERT (ROBERT), GPT-x, embeddings from language models (ELMO)), named entity recognition (NER) (e.g., using Flair®, spaCy®, etc.) and/or any other suitable techniques to determine speech objects (key phrases, topics, etc.).

The system may determine weights (e.g., reflecting relevance, significance, and/or any other suitable properties or characteristics) of the extracted speech objects. The system may use recency of correlated factors and/or relevance previously assigned to the factors to determine the present relevance. For example, the system may correlate a concept of a particular speech object to one or more previously determined features (e.g., symptoms, diagnoses, conditions, events, physiological or behavior patterns, etc.) associated with the individual (e.g., from past encounters). In the event of sufficient correlation (e.g., above a threshold), the system may retrieve (e.g., within the computing environment 100) relevant data from PRI or add relevant data to PRI. For example, an individual mentioning sleep patterns may cause the system to determine significance (e.g., the individual mentioned sleep patterns in a previous encounter) and retrieve relevant data. The provider may ask the individual for permission to upload sleep data (e.g., from individual's device) into PRI if the sleep data is missing. In some examples, the system may use the provider's choosing to retrieve data based on the object of speech as an indication of high relevance. Additionally or alternatively, the system may use the provider's choosing to ignore an object of speech, particularly when repeated, as an indication of low relevance.

At block 440 the system implementing the method 400 determines, by accessing one or more data sets associated with the individual, and based at least in part on the first text and a first object of the one or more objects, a first one or more factors associated with the individual. The data sets may be stored at a PRI server, connected servers, devices of the individual, etc., as discussed above with reference to method 300 and the computing environment 100. In some embodiments, the system omits from the factors any factor already included in the information associated with the previously obtained text.

Continuing the example above, the system may determine that sleep data is relevant, and the first text obtained at block 410 may include a description of a temporal pattern indicating reduced sleep. In response, the system may obtain from available data sets information about room temperature, REM cycles, stress levels, device usage, etc. Furthermore, the system may obtain information about possible conditions, medications, etc., that may cause sleep disturbance. The system may obtain at least some of the information based on the analysis of the audio stream. The system may use any of the relevance determination techniques discussed above with reference to method 300, for example, to determine relevant data for retrieval. For example, the system may use a machine-learned model to determine relevance. That is, the machine-learned model may be used in determining, based on the one or more objects of speech, factors that are associated with the individual and relevant to the encounter (e.g., a symptom that gave rise to the encounter).

At block 450, the method 400 includes generating, based at least in part on the first one or more factors, second text indicative of an update to the first information. Continuing the example above, the system implementing the method 400 may add text describing sleep parameters (e.g., REM, motion, room temperature, ambient light, etc.), possible causes (e.g., travel, medicine that may affect sleep, disorders, etc.), and/or other suitable information. The system may use any of the techniques discussed above with reference to method 300 to generate the text, for example.

The system may use a GUI to prompt a provider to confirm and/or reject newly generated text items. In some examples, confirmations and/or rejections by a provider may form labels and/or other training data for training a machine-learned model that the system uses to determine relevant information (i.e., relevant factors) at block 440. To that end, the system implementing the method 400 may present on a display one or more user controls (e.g., elements 230a-c, etc.) for collecting user input on relevance.

### EXAMPLES

### Example 1

A computer-implemented method comprising: obtaining, by one or more processors, first data indicative of at least a first symptom associated with an encounter between an individual and a provider; obtaining, by the one or more processors, second data comprising sensor data generated by one or more devices associated with the individual; generating, by the one or more processors and based at least in part on the second data, a data set comprising data indicative of one or more temporal patterns in the second data; determining, by the one or more processors and based at least in part on the data set, a first temporal pattern, of the one or more temporal patterns in the second data, that is relevant to the first symptom; and generating, by the one or more processors, output text comprising first text indicative of the first temporal pattern.

### Example 2

The computer-implemented method of Example 1, wherein determining the first temporal pattern that is relevant to the encounter comprises: determining one or more candidate causes of the first symptom by accessing a knowledge base; and determining the first temporal pattern that is relevant to the encounter based at least in part on the data set and the one or more candidate causes.

### Example 3

The computer-implemented method of Example 1, wherein determining the first temporal pattern that is relevant to the encounter comprises: determining the first temporal pattern that is relevant to the encounter based at least in part on one or more factors linked to the first symptom by a machine learned model.

### Example 4

The computer-implemented method of Example 3, wherein the machine learned model comprises a regression model or a tree-based algorithm.

### Example 5

The computer-implemented method of Example 3, wherein the machine learned model comprises a neural network having one or more respective feature weights for the one or more factors.

### Example 6

The computer-implemented method of Example 3, wherein the machine learned model comprises a predictive model trained on a graph having (i) a first node representing the first symptom, (ii) one or more respective nodes representing the one or more factors, and (iii) edges that connect node pairs within the graph and are associated with weights indicative of relevancy between nodes of the node pairs.

### Example 7

The computer-implemented method of Example 1, wherein the first data comprises a predefined code associated with the first symptom.

### Example 8

The computer-implemented method of Example 1, wherein obtaining the first data comprises: receiving unstructured data specifying a reason for the encounter; and generating the first data by applying one or more automated processing techniques to the unstructured data, the one or more automated processing techniques including one or more of (i) a natural language processing technique, (ii) a computer vision technique, or (iii) an automated speech recognition technique.

### Example 9

The computer-implemented method of Example 1, wherein the sensor data comprises first sensor data that was generated by a first device of the one or more devices and is indicative of values of a first parameter during a first time period, and wherein generating the data set comprises: determining an amount of variation in the values of the first parameter during the first time period relative to a baseline for the individual; and adding information based on values of first parameter to the data set when the amount of variation exceeds a threshold amount.

### Example 10

The computer-implemented method of Example 9, wherein determining the amount of variation in the values of the first parameter during the first time period relative to the baseline for the individual comprises: determining a first mean or median value of the values of the first parameter during the first time period; and determining a difference between the first mean or median value and a baseline mean or median value for the individual.

### Example 11

The computer-implemented method of Example 1, wherein obtaining the second data comprises obtaining one or both of: first sensor data indicative of one or more physiological parameters of the individual; and second sensor data indicative of one or more physical activities of the individual.

### Example 12

The computer-implemented method of Example 1, wherein obtaining the second data comprises obtaining environmental sensor data indicative of one or more environmental factors associated with the individual.

### Example 13

The computer-implemented method of Example 1, wherein: the second data further comprises application data (i) generated by an application installed on a mobile device of the individual and configured to receive manual entries of activities, and (ii) indicative of a first activity of the individual during a first time period; generating the data set comprises determining an amount of variation in the first activity of the individual during the first time period relative to a baseline for the individual; and adding information based on the first activity of the individual during the first time period to the data set when the amount of variation exceeds a threshold amount.

### Example 14

The computer-implemented method of Example 13, wherein determining the amount of variation in the first activity of the individual during the first time period relative to the baseline for the individual comprises: determining whether a frequency of the first activity of the individual during the first time period or an amount of the first activity of the individual during the first time period differs from an earlier frequency or amount, respectively, of the first activity of the individual by more than a threshold amount.

### Example 15

The computer-implemented method of Example 1, further comprising: determining, by the one or more processors and based at least in part on the data set, a second temporal pattern, of the one or more temporal patterns in the second data, that is relevant to the encounter, wherein determining the first temporal pattern that is relevant to the encounter includes determining a first metric indicative of how relevant the first temporal pattern is to the encounter, determining the second temporal pattern that is relevant to the encounter includes determining a second metric indicative of how relevant the second temporal pattern is to the encounter, the output text further comprises second text indicative of the second temporal pattern, and an arrangement of the first text and the second text within the output text is indicative of whether the first temporal pattern or the second temporal pattern is more relevant to the encounter.

### Example 16

The computer-implemented method of Example 1, wherein generating the output text includes using a transformer-based machine learned model to generate the output text.

### Example 17

A system comprising: one or more processors; and one or more memories storing processor-executable instructions that, when executed by the one or more processors, cause the one or more processors to perform the method of any one of Examples 1-16.

### Example 18

One or more non-transitory computer-readable media storing processor-executable instructions that, when executed by one or more processors, cause the one or more processors to perform the method of any one of Examples 1-16.

### Example 19

A computer-implemented method comprising: obtaining, by one or more processors, first text indicative of first information associated with an individual and relevant to a first symptom that triggered an encounter between the individual and a provider; obtaining, by the one or more processors, an audio stream comprising speech of the individual and the provider during the encounter; determining, by the one or more processors and based at least in part on the audio stream, one or more objects of the speech; determining, by the one or more processors accessing one or more data sets associated with the individual, and based at least in part on the first text and a first object of the one or more objects, a first one or more factors associated with the individual; and generating, by the one or more processors and based at least in part on the first one or more factors, second text indicative of an update to the first information.

### Example 20

The computer-implemented method of Example 19, further comprising: determining, by the one or more processors, a weight associated with the first object, at least in part by correlating the first symptom to a past symptom of the individual, wherein generating the second text is further based on the weight associated with the first object.

### Example 21

The computer-implemented method of Example 20, wherein determining the weight associated with the first object further comprises: determining the weight based at least in part on one or both of (i) recency of the past symptom, and (ii) an indication of relevance of the first object to the past symptom.

### Example 22

The computer-implemented method of Example 19, wherein determining the first one or more factors associated with the individual comprises omitting, from the first one or more factors, any factor already indicated in the first information associated with the individual.

### Example 23

The computer-implemented method of Example 19, wherein determining the one or more objects of the speech comprises: converting the audio stream to a speech transcript; and determining the one or more objects of the speech at least in part by processing the speech transcript using a transformer-based machine learned model.

### Example 24

The computer-implemented method of Example 19, wherein: determining the one or more objects of the speech comprises converting the audio stream to a speech transcript; and the first object comprises one or more phrases from the speech transcript.

### Example 25

The computer-implemented method of Example 19, wherein at least obtaining the audio stream, determining the one or more objects of the speech, determining the first one or more factors associated with the individual, and generating the second text occur in real time during the encounter.

### Example 26

The computer-implemented method of Example 19, wherein generating the second text includes using a transformer-based machine learned model to generate the second text.

### Example 27

The computer-implemented method of Example 19, wherein determining the one or more factors includes using a machine learned model trained using training data.

### Example 28

The computer-implemented method of Example 27, further comprising: causing to be presented on a display, by the one or more processors, the second text and one or more user controls, wherein the one or more user controls comprise a first user control that, when selected, indicates a confirmation of relevance of a first factor of the first one or more factors relevant to the first symptom, and wherein the training data is based on the confirmation of relevance.

### Example 29

A system comprising: one or more processors; and one or more memories storing processor-executable instructions that, when executed by the one or more processors, cause the one or more processors to perform the method of any one of Examples 19-28.

### Example 30

One or more non-transitory computer-readable media storing processor-executable instructions that, when executed by one or more processors, cause the one or more processors to perform the method of any one of Examples 19-28.

### Conclusion

Throughout this specification, components, operations, or structures described as a single instance may be implemented as multiple instances. Although individual operations of one or more methods (or processes, techniques, routines, etc.) are illustrated and described as separate operations, two or more of the individual operations may be performed concurrently or otherwise in parallel, and nothing requires that the operations be performed in the order illustrated. Structures and functionality (e.g., operations, steps, blocks) presented as separate components in example configurations may be implemented as a combined structure, functionality, or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, operations, blocks, or instructions. These may constitute and/or be implemented by software (e.g., code embodied on a non-transitory, machine-readable medium), hardware, or a combination thereof. In hardware, the routines, etc., may represent tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware component that operates to perform certain operations as described herein.

In various embodiments, a hardware component may be implemented mechanically or electronically. For example, a hardware component may comprise dedicated circuitry or logic that is permanently configured (e.g., as a special-purpose processor, such as a field programmable gate array (FPGA) or an application-specific integrated circuit (ASIC)) to perform certain operations. A hardware component may also or instead comprise programmable logic or circuitry (e.g., as encompassed within one or more general-purpose processors and/or other programmable processor(s)) that is temporarily configured by software to perform certain operations.

Accordingly, the term “hardware component” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where the hardware components include a general-purpose processor configured using software, the general-purpose processor may be configured as respective different hardware components at different times. Software may accordingly configure a processor, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple of such hardware components exist contemporaneously, communications may be achieved through signal transmission (e.g., over appropriate circuits and buses) that connect the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

As noted above, the various operations of example methods (or processes, techniques, routines, etc.) described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions. The components referred to herein may, in some example embodiments, comprise processor-implemented components.

Moreover, each operation of processes illustrated as logical flow graphs may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

The terms “coupled” and “connected,” along with their derivatives, may be used. In particular embodiments, “connected” may be used to indicate that two or more elements are in direct physical or electrical contact with each other, although the context in the description may dictate otherwise when it is apparent that two or more elements are not in direct physical or electrical contact. “Coupled” may mean that two or more elements are in direct physical or electrical contact. However, “coupled” may also mean that two or more elements are not in direct contact with each other, yet still co-operate, transmit between, or interact with each other.

An algorithm may be considered to be a self-consistent sequence of acts or operations leading to a desired result. These include physical manipulations of physical quantities. Usually, though not necessarily, these quantities take the form of electrical, magnetic, or optical signals capable of being stored, transferred, combined, compared, and otherwise manipulated. These signals are commonly referred to as bits, values, elements, symbols, characters, terms, numbers, flags, or the like. It should be understood, however, that all of these and similar terms are to be associated with the appropriate physical quantities and are merely convenient labels applied to these quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “some embodiments,” “one embodiment,” “an embodiment,” “in some examples,” or variations thereof means that a particular element, feature, structure, characteristic, operation, or the like described in connection with the embodiment is included in at least one embodiment, but not every embodiment necessarily includes the particular element, feature, structure, characteristic, operation, or the like. Different instances of such a reference in various places in the specification do not necessarily all refer to the same embodiment, although they may in some cases. Moreover, different instances of such a reference may describe elements, features, structures, characteristics, operations, or the like be combined in any manner as an embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless the context of use clearly indicates otherwise, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “set” is intended to mean a collection of elements and can be a null set (i.e., a set containing zero elements) or may comprise one, two, or more elements. A “subset” is intended to mean a collection of elements that are all elements of a set, but that does not include other elements of the set. A first subset of a set may comprise zero, one, or more elements that are also elements of a second subset of the set. The first subset may be said to be a subset of the second subset if all the elements of the first subset are elements of the second subset, while also being a subset of the set. However, if all the elements of the second subset are also elements of the first subset (in addition to all the elements of the first subset being elements of the second subset), the first subset and the second subset are a single subset/not distinct.

For the purposes of the present disclosure, the term “a” or “an” entity refers to one or more of that entity. As such, the terms “a” or “an”, “one or more”, and “at least one” can be used interchangeably herein unless explicitly contradicted by the specification using the word “only one” or similar. For example, “a first element” may functionally be interpreted as “a first one or more elements” or a “first at least one element.” Unless otherwise apparent from the context of use, reference in the present disclosure to a same set of “one or more processors” (or a same “plurality of processors,” etc.) performing multiple operations can encompass implementations in which performance of the operations is divided among the processor(s) in any suitable way. For example, “generating, by one or more processors, X; and generating, by the one or more processors, Y” can encompass: (1) implementations in which a first subset of the processors (e.g., in a first computing device) generates X and an entirely distinct, second subset of the processors (e.g., in a different, second computing device) independently generates Y; (2) implementations in which one or more or all of the processor(s) (e.g., one or multiple processors in the same device, or multiple processors distributed among multiple devices) contribute to the generation of X and/or Y; and (3) other variations. This may similarly be applied to any other component or feature similarly recited (e.g., as “a component”, “a feature”, “one or more components”, “one or more features”, “a plurality of components”, “a plurality of features”). Moreover, the performance of certain of the operations may be distributed among the one or more components, not only residing within a single machine, but deployed across a number of machines. The set of components may be located in a single geographic location (e.g., within a home environment, an office environment, a cloud environment). In other example embodiments, the set of components may be distributed across two or more geographic locations. Further, “a machine-learned model”, equivalent terms (e.g., “machine learning model,” “machine-learning model,” “machine-learned component”, “artificial intelligence”, “artificial intelligence component”), or species thereof (e.g., “a large language model”, “a neural network”) may include a single machine-learned model or multiple machine-learned models, such as a pipeline comprising two or more machine-learned models arranged in series and/or parallel, an agentic framework of machine-learned models, or the like.

An “artificial intelligence” or “artificial intelligence component” may comprise a machine-learned model. A machine-learned model may comprise a hardware and/or software architecture having structural hyperparameters defining the model's architecture and/or one or more parameters (e.g., coefficient(s), weight(s), biase(s), activation function(s) and/or action function type(s) in examples where the activation function and/or function type is determined as part of training, clustering centroid(s)/medoid(s), partition(s), number of trees, tree depth, split parameters) determined as a result of training the machine-learned model based at least in part on training hyperparameters (e.g., for supervised, semi-supervised, and reinforcement learning models) and/or by iteratively operating the machine-learned model according to the training hyperparameters(e.g., for unsupervised machine-learned models).

In some examples, structural hyperparameter(s) may define component(s) of the model's architecture and/or their configuration/order, such as, for example, the configuration/order specifying which input(s) are provided to one component and which output(s) of that component are provided as input to other component(s) of the machine-learned model; a number, type, and/or configuration of component(s) per layer; a number of layers of the model; a number and/or type of input nodes in an input layer of the model; a number and/or type of nodes in a layer; a number and/or type of output nodes of an output layer of the model; component dimension (e.g., input size versus output size); a number of trees; a maximum tree depth; node split parameters; minimum number of samples in a leaf node of a tree; and/or the like. The component(s) of the model may comprise one or more activation functions and/or activation function type(s) (e.g., gated linear unit (GLU), such as a rectified linear unit (ReLU), leaky RELU, Gaussian error linear unit (GELU), Swish, hyperbolic tangent), one or more attention mechanism and/or attention mechanism types (e.g., self-attention, cross-attention), nodes and split indications and/or probabilities in a decision tree, and/or various other component(s) (e.g., adding and/or normalization layer, pooling layer, filter). Various combinations of any these components (as defined by the structural hyperparameter(s)) may result in different types of model architectures, such as a transformer-based machine-learned model (e.g., encoder-only model(s), encoder-decoder model(s), decoder-only models, generative pre-trained transformer(s) (GPT(s))), neural network(s), multi-layer perceptron(s), Kolmogorov-Arnold network(s), clustering algorithm(s), support vector machine(s), gradient boosting machine(s), and/or the like. The structural parameters and components a machine-learned model comprises may vary depending on the type of machine-learned model.

Training hyperparameter(s) may be used as part of training or otherwise determining the machine-learned model. In some examples, the training hyperparameter(s), in addition to the training data and/or input data, may affect determining the parameter(s) of the target machine-learned model. Using a different set of training hyperparameters to train two machine-learned models that have the same architecture (i.e., the same structural hyperparameters) and using the same training data may result in the parameters of the first machine-learned model differing from the parameters of the second machine-learned model. Despite having the same architecture and having been trained using the same training data, such machine-learned models may generate different outputs from each other, given the same input data. Accordingly, accuracy, precision, recall, and/or bias may vary between such machine-learned models.

In some examples, training hyperparameter(s) may include a train-test split ratio, activation function and/or activation function type (e.g., in examples like Kolmogorov-Arnold networks (KANs) where the activation function type is determined as part of training from an available set of activation functions and/or limits on the activation function parameters specified by the training hyperparameters), training stage(s) (e.g., using a first set of hyperparameters for a first epoch of training, a second set of hyperparameters for a second epoch of training), a batch size and/or number of batches of data in a training epoch, a number of epochs of training, the loss function used (e.g., L1, L2, Huber, Cauchy, cross entropy), the component(s) of the machine-learned model that are altered using the loss for a particular batch or during a particular epoch of training (e.g., some components may be “frozen,” meaning their parameters are not altered based on the loss), learning rate, learning rate optimization algorithm type (e.g., gradient descent, adaptive, stochastic) used to determine an alteration to one or more parameters of one or more components of the machine-learned model to reduce the loss determined by the loss function, learning rate scheduling, and/or the like.

In some examples, the structural hyperparameters and/or the training hyperparameters may be determined by a hyperparameter optimization algorithm or based on user input, such as a software component written by a user or generated by a machine-learned model. The machine-learned model may include any type of model configured, trained, and/or the like to generate a prediction output for a model input. In some examples, any of the logic, component(s), routines, and/or the like discussed herein may be implemented as a machine-learned model.

The machine-learned model may include one or more of any type of machine-learned model including one or more supervised, unsupervised, semi-supervised, and/or reinforcement learning models. Training a machine-learned model may comprise altering one or more parameters of the machine-learned model (e.g., using a loss optimization algorithm) to reduce a loss. Depending on whether the machine-learned model is supervised, semi-supervised, unsupervised, etc. this loss may be determined based at least in part on a difference between an output generated by the model and ground truth data (e.g., a label, an indication of an outcome that resulted from a system using the output), a cost function, a fit of the parameter(s) to a set of data, a fit of an output to a set of data, and/or the like. In some examples, determining an output by a machine-learned model may comprise executing a set of inference operations executed by the machine-learned model according to the target machine-learned model's parameter(s) and structural hyperparameter(s) and using/operating on a set of input data.

Moreover, any discussion of receiving data associated with an individual that may be protected, confidential, or otherwise sensitive information, is understood to have been preceded by transmitting a notice of use of the data to a computing device, account, or other identifier (collectively, “identifier”) associated with the individual, receiving an indication of authorization to use the data from the identifier, and/or providing a mechanism by which a user may cause use of the data to cease or a copy of the data to be provided to the user.

Upon reading this disclosure, those of skill in the art will appreciate still additional alternative structural and functional designs through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S.C. § 112(f) unless traditional means-plus-function language is expressly recited, such as “means for” or “step for” language being explicitly recited in the claim(s).

1. A computer-implemented method comprising:
obtaining, by one or more processors, first text indicative of first information associated with an individual and relevant to a first symptom that triggered an encounter between the individual and a provider;
obtaining, by the one or more processors, an audio stream comprising speech of the individual and the provider during the encounter;
determining, by the one or more processors and based at least in part on the audio stream, one or more objects of the speech;
determining, by the one or more processors accessing one or more data sets associated with the individual, and based at least in part on the first text and a first object of the one or more objects, a first one or more factors associated with the individual; and
generating, by the one or more processors and based at least in part on the first one or more factors, second text indicative of an update to the first information.

2. The computer-implemented method of claim 1, further comprising:
determining, by the one or more processors, a weight associated with the first object, at least in part by correlating the first symptom to a past symptom of the individual,
wherein generating the second text is further based on the weight associated with the first object.

3. The computer-implemented method of claim 2, wherein determining the weight associated with the first object further comprises:
determining the weight based at least in part on one or both of (i) recency of the past symptom, and (ii) an indication of relevance of the first object to the past symptom.

4. The computer-implemented method of claim 1, wherein determining the first one or more factors associated with the individual comprises omitting, from the first one or more factors, any factor already indicated in the first information associated with the individual.

5. The computer-implemented method of claim 1, wherein determining the one or more objects of the speech comprises:
converting the audio stream to a speech transcript; and
determining the one or more objects of the speech at least in part by processing the speech transcript using a transformer-based machine learned model.

6. The computer-implemented method of claim 1, wherein:
determining the one or more objects of the speech comprises converting the audio stream to a speech transcript; and
the first object comprises one or more phrases from the speech transcript.

7. The computer-implemented method of claim 1, wherein at least obtaining the audio stream, determining the one or more objects of the speech, determining the first one or more factors associated with the individual, and generating the second text occur in real time during the encounter.

8. The computer-implemented method of claim 1, wherein generating the second text includes using a transformer-based machine learned model to generate the second text.

9. The computer-implemented method of claim 1, wherein determining the one or more factors includes using a machine learned model trained using training data.

10. The computer-implemented method of claim 9, further comprising:
causing to be presented on a display, by the one or more processors, the second text and one or more user controls,
wherein the one or more user controls comprise a first user control that, when selected, indicates a confirmation of relevance of a first factor of the first one or more factors relevant to the first symptom, and
wherein the training data is based on the confirmation of relevance.

11. A system comprising:
one or more processors; and
one or more memories storing processor-executable instructions that, when executed by the one or more processors, cause the one or more processors to perform operations comprising:
obtaining first text indicative of first information associated with an individual and relevant to a first symptom that triggered an encounter between the individual and a provider;
obtaining an audio stream comprising speech of the individual and the provider during the encounter;
determining, based at least in part on the audio stream, one or more objects of the speech;
determining, by accessing one or more data sets associated with the individual, and based at least in part on the first text and a first object of the one or more objects, a first one or more factors associated with the individual; and
generating, based at least in part on the first one or more factors, second text indicative of an update to the first information.

12. The system of claim 11, wherein the processor-executable instructions, when executed by the one or more processors, cause the one or more processors to perform a further operation comprising:
determining a weight associated with the first object, at least in part by correlating the first symptom to a past symptom of the individual,
wherein generating the second text is further based on the weight associated with the first object.

13. The system of claim 12, wherein the processor-executable instructions, when executed by the one or more processors, cause the one or more processors to perform the determining the weight associated with the first object at least in part by:
determining the weight based at least in part on one or both of (i) recency of the past symptom, and (ii) an indication of relevance of the first object to the past symptom.

14. The system of claim 11, wherein the processor-executable instructions, when executed by the one or more processors, cause the one or more processors to perform the determining the first one or more factors associated with the individual at least in part by:
omitting, from the first one or more factors, any factor already indicated in the first information associated with the individual.

15. The system of claim 11, wherein the processor-executable instructions, when executed by the one or more processors, cause the one or more processors to perform the determining the one or more objects of the speech at least in part by:
converting the audio stream to a speech transcript; and
determining the one or more objects of the speech at least in part by processing the speech transcript using a transformer-based machine learned model.

16. The system of claim 11, wherein:
the processor-executable instructions, when executed by the one or more processors, cause the one or more processors to perform the determining the one or more objects of the speech at least in part by converting the audio stream to a speech transcript; and
the first object comprises one or more phrases from the speech transcript.

17. The system of claim 11, wherein the processor-executable instructions, when executed by the one or more processors, cause the one or more processors to perform at least the obtaining the audio stream, the determining the one or more objects of the speech, the determining the first one or more factors associated with the individual, and the generating the second text in real time during the encounter.

18. The system of claim 11, wherein the processor-executable instructions, when executed by the one or more processors, cause the one or more processors to perform the generating the second text at least in part by using a transformer-based machine learned model to generate the second text.

19. The system of claim 11, wherein the processor-executable instructions, when executed by the one or more processors, cause the one or more processors to perform the determining the one or more factors at least in part by using a machine learned model trained using training data.

20. One or more non-transitory computer-readable media storing processor-executable instructions that, when executed by one or more processors, cause the one or more processors to perform operations comprising:
obtaining first text indicative of first information associated with an individual and relevant to a first symptom that triggered an encounter between the individual and a provider;
obtaining an audio stream comprising speech of the individual and the provider during the encounter;
determining, based at least in part on the audio stream, one or more objects of the speech;
determining, by accessing one or more data sets associated with the individual, and based at least in part on the first text and a first object of the one or more objects, a first one or more factors associated with the individual; and
generating, based at least in part on the first one or more factors, second text indicative of an update to the first information.
